(19) United States
(12) Patent Application Publication
(10) Pub. No.: US 2024/0407391 A1
(43) Pub. Date: Dec. 12, 2024

(54) METHODS OF FORMING TEXTURIZED MYCELIUM PRODUCTS WITH COMPRESSION AND/OR HEAT

(71) Applicant: Emergy Inc., Boulder, CO (US)

(72) Inventors: Stefan Klaus BUCHER, Boulder, CO (US); Justin WHITELEY, Lafayette, CO (US); Tracy B. YATES, Arvada, CO (US)

(21) Appl. No.: 18/694,909

(22) PCT Filed: Sep. 23, 2022
(86) PCT No.: PCT/US2022/044597

§ 371 Date: Mar. 22, 2024

Related U.S. Application Data
(60) Provisional application No. 63/247,652, filed on Sep. 23, 2021.
(60) Provisional application No. 63/247,658, filed on Sep. 23, 2021.

(51) Int. Cl. A23J 3/22; A23J 3/20; A23L 3/12; A23L 31/00

(52) U.S. Cl. CPC A23J 3/227; A23J 3/20; A23L 3/12; A23L 31/00

(57) ABSTRACT

A method of forming an edible meat substitute product includes providing a mycelium mass, disposing the mycelium mass in a container, and applying a vacuum to the container. The method further includes heating the mycelium mass to a first temperature above 25° C. to form a textured mycelium product. The texturized mycelium product has a chewiness in a range in of about 500 g/cm² to about 4,500 g/cm² and/or a firmness that is in a range from about 15 kg to about 20 kg.

### TECHNICAL FIELD

This application claims the benefit of U.S. Provisional Application No. 63/247,658, filed on Sep. 23, 2021, and of U.S. Provisional Application No. 63/247,652, filed on Sep. 23, 2021, the contents of each of which are incorporated by reference in their entirety.

The present disclosure relates generally to the field of fungal mycelium based edible meat substitute products.

### BACKGROUND

Demand for edible products that can provide a high protein content which is drawn from a non-animal source is increasing. Driven by increasing awareness of personal health, edible products that include non-animal sourced components such as proteins and fibers are considered as a healthier alternative to animal protein-based products. In particular, there is growing demand for edible meat substitutes that mimic meat in its composition and texture but are composed of non-animal components, which can reduce reliance on animals such as cows, chicken, and pigs, and reduce the carbon footprint posed by such animals. Thus, there is a need for non-animal protein sources that can facilitate large scale production and adoption of non-animal based edible products.

### SUMMARY

Embodiments described herein relate generally to the methods of forming a texturized mycelium product for obtaining edible meat substitute products that resemble animal meat in their texture and morphology.

In some embodiments, a method of forming an edible meat substitute product includes providing a mycelium mass, disposing the mycelium mass in a container, and applying a vacuum to the container. In some embodiments, a method of forming an edible meat substitute product includes providing a mycelium mass, disposing the mycelium mass in a container, applying a vacuum to the container, concentrating said mycelium mass, and changing its texture. In some embodiments, a method of forming an edible meat substitute product includes providing a mycelium mass, disposing the mycelium mass in a container, concentrating said mycelium mass, and changing its texture. Embodiments of the disclosed methods further include heating the mycelium mass to a first temperature that is at least about 25° C., such as a first temperature that is at least about 30° C., at least about 35° C., at least about 40° C., or at least about 45° C. to form a texturized mycelium product. In some embodiments, the first temperature is in a range from about 25° C. to about 75° C. In some embodiments, the first temperature is in a range from about 25° C. to about 50° C. In some embodiments, the first temperature is in a range from about 40° C. to about 75° C. The texturized mycelium product has a chewiness in a range from about 500 g/cm² to about 4500 g/cm², such as from about 1500 g/cm² to about 2000 g/cm² or from about 1800 g/cm² to about 2000 g/cm². Without wishing to be bound by theory, it is believed that, in at least some embodiments, this method irreversibly transforms the texture of the mycelium mass into a softer, less crumbly, less chewy, more cohesive consistency that is closer to the texture of meat and that has a reduced firmness compared to the material before heat treatment. For the avoidance of doubt, it is to be understood that references throughout this disclosure to a “first temperature” do not necessarily imply or require that a mycelium mass is also heated to a second and/or further temperature because, as discussed elsewhere herein, heating to a second and/or further temperature is optional. By way of non-limiting example, firmness can be measured using devices available from Texture Technologies Corp. (Hamilton, MA).

In other embodiments, a method of forming an edible meat substitute product includes providing a mycelium mass, disposing the mycelium mass in a container, and cooling the mycelium mass to below about 25° C., such as to a temperature in a range from about −10° C. to about 20° C. or in a range from about 0° C. to about 20° C. In other embodiments, a method of forming an edible meat substitute product includes providing a mycelium mass, disposing the mycelium mass in a container, cooling the mycelium mass to below about 25° C., such as to a temperature in a range from about −10° C. to about 20° C. or in a range from about 0° C. to about 20° C. and applying a vacuum to the container. In some embodiments, a method of forming an edible meat substitute product includes providing a mycelium mass, disposing the mycelium mass in a container, cooling the mycelium mass to below about 25° C., such as to a temperature in a range from about −10° C. to about 20° C. or in a range from about 0° C. to about 20° C., applying a vacuum to the container, concentrating said mycelium mass, and changing its texture. In some embodiments, a method of forming an edible meat substitute product includes providing a mycelium mass, disposing the mycelium mass in a container, cooling the mycelium mass to below about 25° C., such as to a temperature in a range from about −10° C. to about 20° C. or in a range from about 0° C. to about 20° C., concentrating said mycelium mass, and changing its texture.

In other embodiments, a method of forming an edible meat substitute product includes providing a mycelium mass, disposing the mycelium mass in a container, and flowing a gas around the mycelium mass. The gas is at least one of air, carbon dioxide, or nitrogen. The method further includes heating the mycelium mass to a first temperature that is above about 25° C., such as a first temperature that is at least about 30° C., at least about 35° C., at least about 40° C., or at least about 45° C. or a first temperature that is in a range from above about 25° C. to about 50° C., to form a texturized mycelium product having a chewiness in a range from about 500 g/cm² to about 4500 g/cm², such as from about 1500 g/cm² to about 2000 g/cm² or from about 1800 g/cm² to about 2000 g/cm², and a reduced firmness compared to the material before heat treatment. For example, in some embodiments, the firmness of the texturized mycelium product may be from about 40% to about 60% of the firmness of the mycelium mass before heat treatment. In some embodiments, an untreated mycelium mass may have a firmness of greater than or equal to 35 kg, and the texturized mycelium product may have a firmness that is in a range from about 15 kg to about 20 kg.

In some embodiments, the mycelium mass is a nutritionally enhanced mycelium product including Vitamin D and/or iron. In some embodiments, the mycelium mass is a nutritionally enhanced mycelium product having a vitamin D content of greater than 1 μg per 100 grams of the enhanced mycelium product and/or an iron content greater than or equal to 5 mg of iron per 100 g of mycelium mass. In some embodiments, the mycelium mass has an iron content of from about 5 mg of iron per 100 g of mycelium mass to about 15 mg of iron per 100 g of mycelium mass and a vitamin D content greater than about 1 microgram per 100 grams of the enhanced mycelium product.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a flow chart of a method for forming a texturized mycelium product, in accordance with some embodiments.

FIG. 2 is a flow chart of another method for forming a texturized mycelium product, in accordance with some embodiments.

FIG. 3A is an image of a mycelium mass before texturization, in accordance with some embodiments.

FIG. 3B is an image of a texturized mycelium product formed from the mycelium mass after texturization, in accordance with some embodiments.

FIG. 4 is a plot of chewiness of the mycelium mass before and after texturization, in accordance with some embodiments.

FIG. 5 is a graph of the firmness values measured for raw mycelium mass and texturized mycelium product.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to methods of forming a texturized mycelium product for obtaining an edible meat substitute product that resembles animal meats in their texture and morphology. Particularly, various embodiments described herein provide methods of providing a mycelium mass and disposing the mycelium mass in a container. In some embodiments, the methods include applying a vacuum to the container. In other embodiments, the methods may include flowing a gas around the mycelium mass. The gas may be at least one of air, carbon dioxide, or nitrogen. Applying a vacuum to the mycelium mass or flowing a gas around the mycelium mass may texturize the mycelium mass so as to form a texturized mycelium product, providing an irreversible change in the texture and a product that more closely resembles animal meats. In some embodiments, heat may also be applied at a first temperature that is at least about 25° C., such as a first temperature that is at least about 30° C., at least about 35° C., at least about 40° C., or at least about 45° C. or a first temperature that is in a range from 25° C. to 50° C. to the mycelium mass in order to form a texturized mycelium product having a chewiness in a range from about 500 g/cm² to about 4500 g/cm², such as from about 1500 g/cm² to about 2000 g/cm² or from about 1800 g/cm² to about 2000 g/cm², and to further texturize the fungal mycelium. In some embodiments, at least one food additive or color may be added to the mycelium mass. In some embodiments, the mycelium mass is a nutritionally enhanced mycelium product including Vitamin D and/or iron. In some embodiments, the mycelium mass is a nutritionally enhanced mycelium product having a vitamin D content of greater than 1 μg per 100 grams of the enhanced mycelium product and/or an iron content greater than or equal to 5 mg of iron per 100 g of mycelium mass. In some embodiments, the mycelium mass has an iron content of from about 5 mg of iron per 100 g of mycelium mass to about 15 mg of iron per 100 g of mycelium mass.

One parameter in forming edible meat substitute products that include fungal mycelium is the texture of the product. Achieving a texture that resembles real meat (e.g., a beef steak, a chicken breast piece, etc.) is important for such meat substitute products in becoming a viable replacement for real meat products. Heating mycelium mass may be a viable method of texturizing such masses to form mycelium product. However, direct exposure of a pre-texture mass to a liquid source of heat (e.g., heated water) can lead to disintegration of the mycelium mass, which is undesirable. Previous methods for texturizing mycelium masses may include removing moisture in the mass through dehydration and adding the water back in which may lead to more processing steps or drying the mycelium mass in a convection oven which may lead to longer processing times such as a minimum of four hours.

Various embodiments of the methods of forming a texturized mycelium product by texturizing mycelium may provide one or more benefits including, for example: (1) providing an irreversible change in the texture of the texturized mycelium product; (2) providing a continuously solid structure; (3) providing a more efficient process of texturizing the mycelium mass; (4) removing undesired flavors such as undesired enzymes, such as by denaturing enzymes that may generate undesired flavors; (5) arresting the growth and metabolism of the mycelium such as by deactivating heat-sensitive growth inhibitors and/or reducing antinutritional factors by heat denaturization; (6) providing a process for pasteurization; (7) increasing chewiness and/or reducing firmness and crumbliness of the mycelium mass; (8) lowering protein solubility; (9) increasing digestibility; (9) reducing the microbial load of the mycelium mass; (10) eliminating a rehydrating step in a standard compression process for mycelium masses; (11) providing a more desirable meat alternative for consumers; and (12) reducing the processing time from at least four hours to less than one hour.

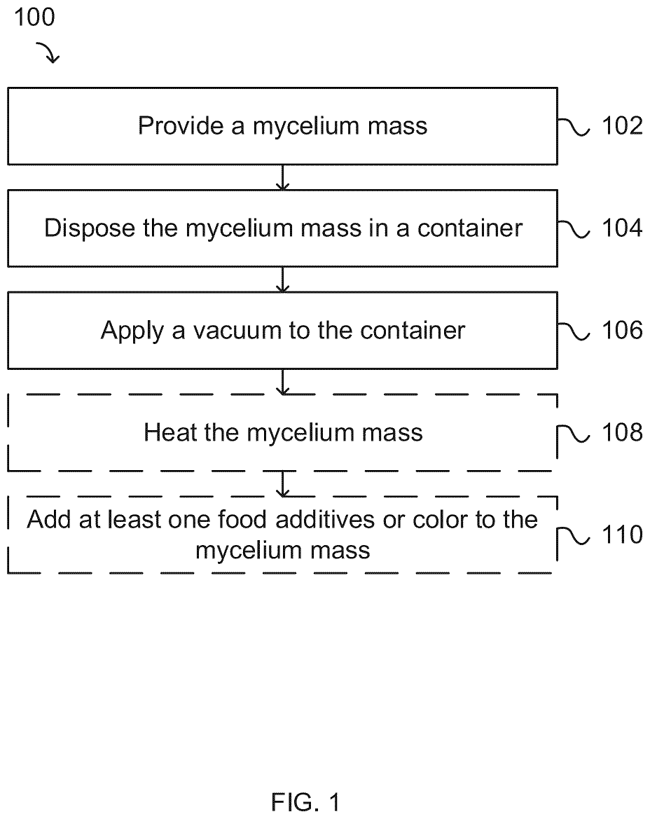

FIG. 1 illustrates a block diagram of an example method 100 for forming an edible meat substitute product, according to an embodiment. In a brief overview, the method 100 may include providing a mycelium mass, at 102. The method 100 may include disposing the mycelium mass in a container, at 104. The method 100 may include applying a vacuum to the container, at 106. The method 100 may include heating the mycelium mass, at 108. The method 100 may include adding at least one food additive or color to the mycelium mass, at 110.

In further detail, the method 100 may include providing a mycelium mass, at 102. The mycelium mass may be fungal mycelium and can include fungi from Ascomycota and Zygomycota, including the genera Aspergillus, Fusarium, Neurospora, and Monascus. Other species can include edible varieties of division Basidiomycota and generus Lentinula. One genus is Neurospora, which is used in food production through solid fermentation. The genus of Neurospora are known for highly efficient biomass production as well as ability to break down complex carbohydrates. For certain species of Neurospora, no known allergies have been detected and no levels of mycotoxins are produced. In some embodiments, the fungal mycelium comprises fungi from the genus Neurospora. In some embodiments, the fungal mycelium consists of fungi from the genus Neurospora. In some embodiments, the fungal mycelium comprises fungi from the species Neurospora crassa. In some embodiments, the fungal mycelium consists of fungi from the species Neurospora crassa. In addition to monocultures of filamentous fungi, multiple strains can be cultivated at once to tune the protein, amino acid, mineral, texture, and flavor profiles of the final biomass. The aforementioned species of fungi do not inherently have vitamin D.

Methods of growing fungal cells to produce mycelium are known in the art and include, as non-limiting examples, methods described in U.S. Patent Application Publication No. 2019/0373935 A1 (entitled “METHOD FOR GROWING FUNGAL MYCELIUM AND FORMING EDIBLE PRODUCTS THEREFROM”), the content of which is herein incorporated by reference, specifically in the passages reproduced below.

In one example, Neurospora crassa (N. crassa) was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 2 g/L potassium phosphate monobasic, 1 g/L sodium nitrate, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9.5 g/L. Protein analysis yields a crude protein content of 57 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9 g/L. Protein analysis yields a crude protein content of 55 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 30 g/L sucrose, 3 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 11 g/L. Protein analysis yields a crude protein content of 63 wt %. Amino acid analysis yields a PDCAAS score of 1.0 for the fibrous mycelium mass. The fibrous mycelium mass has a combined methionine and cysteine content of 27 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 3.25 g/L urea, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 8.5 g/L. Protein analysis yields a crude protein content of 56 wt %. Amino acid analysis yields a PDCAAS score of 1.0. The fibrous mycelium mass has a combined methionine and cysteine content of 25 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 15% ammonium hydroxide buffer. After 24 hours, the mycelium is harvested using a cheese cloth, dewatered in a cider press, and completely dried in a dehydrator set at 74° C. The total cell dry weight is 10 g/L. Protein analysis yields a crude protein content of 60 wt %. Amino acid analysis yields a PDCAAS score of 1.0. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 12 hours, 10 g/L sucrose and 1 g/L ammonium nitrate is added to the system. After 24 hours total, the mycelium is harvested using a cheese cloth, dewatered in a cider press, and completely dried in a dehydrator set at 74° C. The total cell dry weight is 12 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. Following straining with cheese cloth and pressing, all media is collected, autoclaved and reused by only adding 20 g/L sucrose, 2 g/L ammonium nitrate, and 1 g/L potassium phosphate monobasic. The repeated fed-batch process is carried out for 60 hours total. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

The fungal cells may be grown to produce mycelium in a growth media which may include nutrients such as sugar, a nitrogen-containing compound, and a phosphate-containing compound in a broth. The sugar can be in the range from about 5 to about 50 g/L (e.g., 5 g/L, 10 g/L, 20 g/L, 30 g/L, 40 g/L, or 50 g/L, inclusive). The sugar can include sucrose, glucose, fructose, molasses, or a mixture of sugars. The nitrogen-containing compound can be in the range from about 0.5 to about 10 g/L (e.g., 0.5 g/L, 1 g/L, 2 g/L, 3 g/L, 4 g/L, 5 g/L, or 10 g/L, inclusive). The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range from about 0.1 to about 5 g/L (e.g., 0.1 g/L, 0.2 g/L, 0.3 g/L, 0.4 g/L, 0.5 g/L, 1 g/L, 2 g/L, 3 g/L, 4 g/L, or 5 g/L, inclusive). The phosphate-containing compound can be potassium phosphate, sodium phosphate, phosphoric acid, or a mixture of phosphate-containing compounds.

In some embodiments, methods of growing fungal cells to produce mycelium include providing a first growth media having a first iron amount in a range from 0.1 mg/L to 10 mg/L. Such methods may include growing fungal cells in the first growth media so as to produce a mycelium mass having an iron content. Such methods may include separating the mycelium mass from the first growth media. In further detail, such methods may include providing a first growth media having a first iron amount in a range from 0.1 mg/L to 10 mg/L, at 202. The first growth media may have a first iron amount in a range from about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the first iron amount is in a range from about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The first iron amount may be adjusted according to the desired iron content. The first iron amount may be provided by any suitable iron-containing material, for example, iron salts (e.g., ferric citrate, ferrous sulfate, ferrous gluconate, ferrous fumarate, ferric sulfate, ammonium ion sulfate, heme iron polypeptides, carbonyl iron, etc.).

Growing the mycelium mass in a first growth media with an increased iron content may allow the mycelium mass to accumulate the iron intracellularly so as to boost the iron content of the mycelium mass without fortification with added salts or additives after the final product has been formed. In some embodiments, the mycelium mass may meet or exceed the RDA for iron, which is 8 milligrams/day for men, for women aged 19-50 years is 18 milligrams/day, and for women aged 51 and older is 8 milligrams/day. In some embodiments, increasing the first iron amount in the first growth media will increase the iron content of the mycelium mass without impacting growth rate.

In some embodiments, a second growth media may be added to the mycelium mass with a second iron amount in a range from about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the second iron amount is in a range from about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The second iron amount may be the same as or different from the first iron amount. In some embodiments, the mycelium mass is in the first growth media for a time period greater than or equal to about 5 hours (e.g., 5 hours, 6 hours, etc.) and the second growth media for a time period greater than or equal to about 30 seconds (e.g., 30 seconds, 60 seconds, 120 seconds, etc.). In some embodiments, the second growth media may be provided to the mycelium mass after the complete removal and separation of the first growth media from the mycelium mass. In some embodiments, the second growth media may be provided after only a partial removal and separation of the first growth media from the mycelium mass. In some embodiments, the second growth media may be provided to the first growth media without removal of the first growth media from the mycelium mass. The amount of the second growth media may be equal to, less than, or greater than the original amount of the first growth media. In some embodiments, adding the second growth media may add more nutrients (e.g., iron) to the mycelium mass. Increasing the amount of iron in the second growth media or the first growth media may increase the iron content in the mycelium mass. In some embodiments, the second growth media may be added at various stages in the formation of the mycelium mass growth such as to a fermentation broth. It is understood that the number of growth media used to grow the mycelium mass is not limited to two, and any suitable number of growth media used to deliver nutrients (e.g., iron) may be used.

In some embodiments, a mycelium mass with an enhanced nutritional content may be formed by a process of providing a first growth media having a first iron amount in a range from 0.1 mg/L to 10 mg/L and growing fungal cells in the first growth media such that the fungal cells produce a mycelium mass. The mycelium mass may have an iron content greater than or equal to 5 mg of iron per 100 g of the mycelium mass. In some embodiments, the mycelium mass has an iron content from about 5 mg of iron per 100 g of mycelium mass to about 15 mg of iron per 100 g of mycelium mass. The process may further include separating the mycelium mass from the first growth media.

The fungal cells can be grown at a temperature in a range from about 25° C. to about 45° C. (e.g., 25° C., 30° C., 35° C., 40° C., or 45° C., inclusive). The fungal cells can be grown in a range from about 12 hours to about 48 hours (e.g., 12 hours, 18 hours, 24 hours, 30 hours, 36 hours, 42 hours, or 48 hours, inclusive). Growing fungal cells can produce a yield of about 5 to about 20 g/L (e.g., 5 g/L, 10 g/L, 15 g/L, or 20 g/L, inclusive) of fungal cell dry weight. The mycelium mass can have a protein content of greater than or equal to about 40 wt % (e.g., 40 wt %, 50 wt %, 60 wt %, 70 wt %, 80 wt %, 90 wt %, or 100 wt %, inclusive) (dry weight). In some embodiments, the mycelium mass may have a protein content of about 50 to about 65% (e.g., 50%, 55%, 60%, or 65%, inclusive) (dry weight). The mycelium mass can have a combined methionine and cysteine content of at least about 25 mg/g crude protein.

In at least some embodiments of growing fungal cells disclosed herein (e.g., growing fungal cells such that the fungal cells produce mycelium), the fungal cells are grown through a submerged fermentation process or in liquid media. Accordingly, embodiments of growing fungal cells (e.g., growing fungal cells such that the fungal cells produce mycelium) described herein are distinguished from processes in which growth occurs on a substrate, such as in solid-state fermentation or surface fermentation.

Fungal cells grown through a submerged fermentation process can be grown either as aggregated masses referred to as pellets, as filamentous mycelium, or as a combination of both pellets and filamentous mycelium. In at least some embodiments of growing fungal cells disclosed herein (e.g., growing fungal cells such that the fungal cells produce mycelium), the fungal cells grow as filamentous mycelium that is free of pellets.

The mycelium mass may then be separated from the growth media. In some embodiments, the mycelium mass may be mixed with food additives or colors. Additives can include vegetable or animal proteins, fats, emulsifiers, thickeners, stabilizers, flavoring, any other suitable additive, or combinations thereof.

In some embodiments, the mycelium mass is mycelium product that has been exposed to light so as to form an enhanced mycelium product having a vitamin D content. In exposing the mycelium product to light, a wavelength of the light, a distance to the light, and an exposure time may be varied. The light may have a wavelength in a range from about 100 to about 400 nanometers (e.g., 100 nanometers, 150 nanometers, 200 nanometers, 250 nanometers, 300 nanometers, 350 nanometers, or 400 nanometers, inclusive). The mycelium product may be exposed to the light for a first period of time. In some embodiments, the light may have a wavelength in the Ultraviolet (UV) radiation region. The UV radiation region may include three types of UV rays such as UVA, UVB, and UVC which all have different wavelengths. In some embodiments, the wavelength may be in the UVA region in a range from about 315 to about 400 nanometers (e.g., 315 nanometers, 325 nanometers, 335 nanometers, 345 nanometers, 355 nanometers, 365 nanometers, 375 nanometers, 385 nanometers, 395 nanometers, or 400 nanometers, inclusive). In some embodiments, the wavelength may be in the UVB region in a range from about 280 to about 315 nanometers (e.g., 280 nanometers, 290 nanometers, 300 nanometers, 310 nanometers, or 315 nanometers, inclusive). In some embodiments, the wavelength may be in the UVC region in a range from about 100 to about 280 nanometers (e.g., 100 nanometers, 120 nanometers, 140 nanometers, 160 nanometers, 180 nanometers, 200 nanometers, 220 nanometers, 240 nanometers, 260 nanometers, or 280 nanometers, inclusive). The light may have any suitable light source such as, but not limited to, a UVA lightbulb, a UVB lightbulb, a UVC lightbulb or, a UV lamp. In some embodiments, the mycelium product may be exposed to light (e.g., UV light) while the mycelium product is disposed in a vessel (e.g., a fermentation tank), such as by positioning a source of light (e.g., UV light) within the vessel containing the mycelium product. In some embodiments, the mycelium product may be wet and exposed to UV radiation in a transparent conduit (e.g., a glass tube, a transparent container having an inlet and an outlet, etc.) as it is pumped from a tank (e.g., a fermentation tank) to processing (e.g., post-harvest processing). In other embodiments, the mycelium product may be exposed to UV light as it travels on a conveyer (e.g., a conveyor belt or chain) at any point of processing up until packaging. Thus, it is to be understood that a source of light (e.g., UV light) may be located in the same space as mycelium product (e.g., over or near a conveyer or within vessel containing mycelium product) or located externally to a vessel containing mycelium product (e.g., outside of a conduit carrying mycelium product).

In at least some embodiments comprising exposing mycelium product to light, the exposure does not cause the production of pigmentation in the mycelium product. In at least some instances, the production of pigmentation in mycelium upon exposing the mycelium to light is undesirable. Accordingly, at least some embodiments comprising exposing mycelium product to light that are described herein constitute an improvement over processes involving exposing mycelium to light that result in the production of pigmentation.

The mycelium product is exposed to the light for a first period of time. In some embodiments, the first period of time is in a range from about 1 seconds to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). In some embodiments, the first period of time is in a range from about 25 to about 45 seconds (e.g., 25 seconds, 30 seconds, 35 seconds, 40 seconds, or 45 seconds, inclusive). In some embodiments, the mycelium product is at a distance from a source of the light. In some embodiments, the distance is in a range from about 1 to about 10 centimeters (e.g., 1 centimeter, 2 centimeters, 3 centimeters, 4 centimeters, 5 centimeters, 6 centimeters, 7 centimeters, 8 centimeters, 9 centimeters, or 10 centimeters, inclusive). In some embodiments, the amount of a UV dose exposed to the mycelium product may be varied based on time and intensity of exposure. In such embodiments, the dose may be modeled by Equation 1.

$$UV Dose = UV Intensity [ \frac{μ W}{{cm}^{2}} ] * Exposure Time [ sec ] \qquad [ 1 ]$$

In some embodiments, exposing the mycelium product to the light comprises exposing a first side of the mycelium product to the light for the first period of time. In such embodiments, a second side of the mycelium product may be exposed to the light for a second period of time after the first time period. For example, the mycelium product may be flipped over to the second side (e.g., a back side, or a side orthogonal to the first side) of the mycelium product, which is then exposed to the light, or moved laterally to align the second side (e.g., a second portion of a top side). The second period of time may be in a range from about 1 to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). The second period of time may be in a range from about 25 to about 45 seconds (e.g., 25 seconds, 30 seconds, 35 seconds, 40 seconds, or 45 seconds, inclusive). The second period of time may be the same as the first period of time, or different than the first period of time. The second side may be on the same surface as the first side, or on a different surface from the first side. The second side may overlap the first side. The second side may have an area that is larger than, less than, or equal to an area of the first side.

The second side may be exposed to the light in the same wavelength range or region (e.g., UVA, UVB, or UVC) as the first side. The second side may be exposed to the light in a different wavelength range or different region as the first side. The second side may be exposed to light from the same source or a different source as the first side. The second side may be at a distance from the source of light in a range from about 1 to about 10 centimeters (1 centimeters, 2 centimeters, 3 centimeters, 4 centimeters, 5 centimeters, 6 centimeters, 7 centimeters, 8 centimeters, 9 centimeters, or 10 centimeters, inclusive). The second side may be at the same distance or a different distance from the source of light as the first side.

Exposing the mycelium product to the light (e.g., UV radiation) may cause intracellular production of vitamin D in the mycelium product so as to produce an enhance mycelium product with a vitamin D content. Thus, unnatural fortification of the mycelium product by separately adding vitamin D thereto, is not required. The mycelium product may contain ergosterol, a vitamin D precursor, which may be converted to vitamin D2 by exposure to UV light.

The vitamin D content of the mycelium product after exposure to the light (e.g., UV light) may be greater than or equal to about 1 microgram per 100 grams of the enhanced mycelium product (e.g., 1 microgram per 100 grams, 2 micrograms per 100 grams, 3 micrograms per 100 grams, 4 micrograms per 100 grams, 5 micrograms per 100 grams, etc.). In some embodiments, the vitamin D content may meet or exceed the RDA for vitamin D in the enhanced mycelium product. The RDA for vitamin D for children 1 to 18 years old, inclusive, may be 15 micrograms. The RDA for vitamin D for adults 19 to 70 years old, inclusive, may be 15 micrograms. The RDA for vitamin D for adults 71 years and older may be 20 micrograms. The RDA for vitamin D for pregnant and breastfeeding women may be 15 micrograms. Thus, in some embodiments, the vitamin D content of the mycelium product after exposure to the light (e.g., UV light) may be about 15 micrograms per 100 g of enhanced mycelium product or about 20 micrograms per 100 g of enhanced mycelium product. In some embodiments, the vitamin D content of the mycelium product after exposure to the light (e.g., UV light) may be greater than about 15 micrograms per 100 g of enhanced mycelium product or greater than about 20 micrograms per 100 g of enhanced mycelium product.

In some embodiments, the vitamin D content of the mycelium product after exposure to the light (e.g., UV light) does not exceed the tolerable daily upper intake level of vitamin D. The tolerable upper intake level of vitamin D for individuals aged 9 years and older may be 100 micrograms. Thus, in some embodiments, the vitamin D content of the mycelium product after exposure to the light (e.g., UV light) may be about 100 micrograms or less per 100 g of enhanced mycelium product.

In some embodiments, the vitamin D content of the mycelium product after exposure to the light (e.g., UV light) may be from about 1 microgram per 100 grams of the enhanced mycelium product to about 1 milligram (i.e., 1,000 micrograms) per 100 grams of the enhanced mycelium product. For example, in some embodiments, the vitamin D content of the mycelium product after exposure to the light (e.g., UV light) may be from about 1 microgram per 100 grams of the enhanced mycelium product to about 100 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 200 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 300 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 400 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 500 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 600 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 700 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 800 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 900 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 5 micrograms per 100 grams of the enhanced mycelium product, from about 1 microgram per 100 grams of the enhanced mycelium product to about 10 micrograms per 100 grams of the enhanced mycelium product, from about 1 microgram per 100 grams of the enhanced mycelium product to about 15 micrograms per 100 grams of the enhanced mycelium product, or from about 1 microgram per 100 grams of the enhanced mycelium product to about 20 micrograms per 100 grams of the enhanced mycelium product, such as about 1 microgram per 100 grams of the enhanced mycelium product, about 2 micrograms per 100 grams of the enhanced mycelium product, about 3 microgram per 100 grams of the enhanced mycelium product, about 4 micrograms per 100 grams of the enhanced mycelium product, about 5 micrograms per 100 grams of the enhanced mycelium product, about 6 micrograms per 100 grams of the enhanced mycelium product, about 7 micrograms per 100 grams of the enhanced mycelium product, about 8 micrograms per 100 grams of the enhanced mycelium product, about 9 micrograms per 100 grams of the enhanced mycelium product, about 10 micrograms per 100 grams of the enhanced mycelium product, about 11 micrograms per 100 grams of the enhanced mycelium product, about 12 micrograms per 100 grams of the enhanced mycelium product, about 13 micrograms per 100 grams of the enhanced mycelium product, about 14 micrograms per 100 grams of the enhanced mycelium product, about 15 micrograms per 100 grams of the enhanced mycelium product, about 16 micrograms per 100 grams of the enhanced mycelium product, about 17 micrograms per 100 grams of the enhanced mycelium product, about 18 micrograms per 100 grams of the enhanced mycelium product, about 19 micrograms per 100 grams of the enhanced mycelium product, or about 20 micrograms per 100 grams of the enhanced mycelium product.

In some embodiments, the mycelium product may be exposed to the UV light for a period of time outside of the range from 1 to 300 seconds. In such embodiments, the vitamin D content may be greater than about 100 micrograms per 100 grams of the enhanced mycelium product (e.g., greater than 1,000 micrograms per 100 grams of the enhanced mycelium product.) In such embodiments, an excessive amount of vitamin D may be toxic to humans, for examples, who are mildly vitamin D sufficient. However, such high vitamin D content enhanced mycelium may be used to produce mycelium based edible meat substitutes for animals or severely deficient humans who need a high dose of vitamin D so as to increase their blood vitamin D levels to a normal range in a short amount of time. Such high vitamin D content enhanced mycelium may also be used to produce a vitamin D supplement.

In some embodiments, a mycelium mass with an enhanced nutritional content may be formed by a method that increases both the vitamin D content and the iron content of an enhanced mycelium product.

In some embodiments, exposure of the mycelium product to light (e.g., UV light) may induce intracellular production of vitamin D in the mycelium product to such an extent so as to produce an enhanced mycelium product with a vitamin D content that renders the enhanced mycelium product unfit for human consumption (for example, because its vitamin D content is so high as to create a risk of vitamin D toxicity if consumed by humans). The present disclosure provides approaches to address that concern. In some embodiments, the vitamin D content in an enhanced mycelium product is controlled (e.g., to ensure vitamin D levels that are appropriate for human consumption) by moderating the duration of exposure of the mycelium product to light (e.g., UV light) and/or by moderating the wavelength and/or intensity of the light source. In some embodiments, the duration of exposure of the mycelium product to light (e.g., UV light) is moderated by adjusting and/or controlling the flow rate of a fluid phase containing mycelium product as it is exposed to light (e.g., UV light). In other embodiments, only a portion of a quantity of mycelium product is exposed to light (e.g., UV light) that induces intracellular production of vitamin D in the mycelium product so as to produce an enhanced mycelium product, and that portion is mixed with additional amounts of non-irradiated mycelium product. In some embodiments, the admixture of a portion of mycelium product exposed to light (e.g., UV light) and an amount of non-irradiated mycelium product has, as a whole, a vitamin D content that is low enough to be suitable for human consumption. For example, in some embodiments, such an admixture has a vitamin D content from about 1 microgram per 100 grams of the enhanced mycelium product to about 20 micrograms per 100 grams of the enhanced mycelium product, such as from about 1 microgram per 100 grams of the enhanced mycelium product to about 10 micrograms per 100 grams of the enhanced mycelium product or from about 1 microgram per 100 grams of the enhanced mycelium product to about 15 micrograms per 100 grams of the enhanced mycelium product.

In some embodiments where only a portion of a quantity of mycelium product is exposed to light (e.g., UV light) that induces intracellular production of vitamin D in the mycelium product so as to produce an enhanced mycelium product, that portion of the quantity of mycelium product is in a fluid phase and is routed to an apparatus containing a light source (e.g., a UV light source, such as a UVB light source), exposed to light (e.g., UV light), and subsequently combined with remaining non-irradiated mycelium product in a fluid phase. For example, in an embodiment, a portion of a fluid phase containing mycelium is routed from a fermentation tank or other vessel to an apparatus containing a light source (e.g., a UV light source, such as a UVB light source), exposed to light, and recombined with non-irradiated mycelium product in a fluid phase. In some such embodiments, the irradiated portion of the fluid phase is recombined with non-irradiated mycelium product through a static mixture to provide a uniform dispersion of irradiated and non-irradiated mycelium product. In some embodiments, the apparatus containing a light source is a modified version of a device used for sanitizing water with light (e.g., UV light), such as a Sanitron® device. In some embodiments, the amount of fluid-phase mycelium product that is routed to the apparatus and exposed to light (e.g., UV light) is from about 1/40^{th} to about 1/10^{th} of the total amount of mycelium product prepared, such as about 1/40^{th}, 1/35^{th}, 1/30^{th}, 1/25^{th}, 1/20^{th}, 1/15^{th}, or 1/10^{th} of the total amount of mycelium product prepared. For example, in an embodiment where the amount of fluid-phase mycelium product that is routed to the apparatus and exposed to light (e.g., UV light) is 1/10^{th} of the total amount of mycelium product prepared, it is to be understood that 1/10^{th} of the entire volume of fluid phase mycelium product is routed to the apparatus and exposed to light (e.g., UV light), with 9/10^{th} of the entire volume of fluid phase mycelium product remaining non-irradiated. The 1/10^{th} of the entire volume of fluid phase mycelium product that was exposed to light (e.g., UV light) is recombined with the remaining 9/10^{th} of the entire volume of fluid phase mycelium product, providing a volume of fluid phase mycelium product that, as a whole, has a higher vitamin D content compared to the vitamin D content of the volume of fluid phase mycelium product prior to irradiation of the portion of fluid phase mycelium product. In some embodiments, the light source in the apparatus is a UVB light source. In some embodiments, the light source in the apparatus is a UVB light source emitting UVB light having a wavelength that is from about 280 nanometers to about 315 nanometers.

The method 100 include may include disposing the mycelium mass in a container, at 104, in some embodiments. The container may include plastic, such as but not limited to, a plastic bag. In such embodiments, the plastic may comprise low density polyethylene, high density polyethylene, polypropylene, polyethylene terephthalate, silicone, or any other suitable polymer. The container may include any other suitable material such as metal, ceramic, or a composite. The container may be enclosed. The container may be a mold. The mold can be of various shapes and sizes such as, but not limited to, rectangular, cubic, trapezoidal, triangular, or spherical. For example, the mold can have sidewalls extending from the base. The sidewalls can hold the mycelium mass inside the mold. The mold can be flexible. The mold may have a removable lid configured to selectively enclose the mold. The mold may have complex shapes such as shapes that take the form of animal meats such as chicken breast, beef steaks, pork chops, or any other suitable shape. The mold may be solid or semi-solid.

The method 100 may include applying a vacuum to the container, at 106. For example, the vacuum may be applied by any suitable device configured to provide a vacuum, such as a pump (e.g., a rotary vane pump, a diaphragm pump, a piston pump, a scroll pump, a screw pump, a Wankel pump, an external vane pump, any positive displacement pump, or any other suitable pump). The pump may be fluidly coupled with the container and configured to seal the container and remove air from the container. The vacuum may be applied by any other suitable device and any other suitable method. The applied vacuum may be an imperfect vacuum. The vacuum may be applied to the container for a first vacuum time period in a range from about 1 to about 150 minutes (e.g., 1 minutes, 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, or 150 minutes, inclusive). The applied vacuum may arrest the growth and/or metabolism of the mycelium mass disposed in the container, reducing the development of undesired flavors.

The method 100 may include heating the mycelium mass to a first temperature and optionally, a second temperature, to form the texturized mycelium product, at 108. The container and/or the mycelium mass may be heated to a first temperature that is at least about 25° C., such as a first temperature that is at least about 30° C., at least about 35° C., at least about 40° C., or at least about 45° C. or a first temperature that is in a range from at least about 25° C. to about 50° C. (e.g., 25° C., 30° C., 35° C., 40° C., 45° C., or 50° C. inclusive), so as to form a texturized mycelium product with a chewiness. The texturized mycelium product may have a chewiness in a range from about 500 to about 4500 g/cm² (e.g., 500 g/cm², 1000 g/cm², 1500 g/cm², 1600 g/cm², 1700 g/cm², 1800 g/cm², 1900 g/cm², 2000 g/cm², 2100 g/cm², 2200 g/cm², 2300 g/cm², 2400 g/cm², 2500 g/cm², 3000 g/cm², 3500 g/cm², 4000 g/cm², or 4500 g/cm², inclusive), such as from about 1500 g/cm² to about 2000 g/cm² or from about 1800 g/cm² to about 2000 g/cm² . . . . A mycelium mass that does not undergo texturization may have a chewiness in a range from about 1000 to about 1500 g/cm² (e.g., 1000 g/cm², 1100 g/cm², 1200 g/cm², 1300 g/cm², 1400 g/cm², or 1500 g/cm², inclusive) in comparison. The texturized mycelium product may have a reduced firmness compared to the untreated mycelium mass before heat treatment. For example, in some embodiments, the firmness of the texturized mycelium product may be from about 40% to about 60% of the firmness of the untreated mycelium mass before heat treatment. In some embodiments, an untreated mycelium mass may have a firmness of greater than or equal to 35 kg, and the texturized mycelium product may have a firmness that is in a range from about 15 kg to about 20 kg.

Applying the vacuum to the container and/or heating the container to the first temperature may form a texturized mycelium product and texturize the mycelium mass. Texturization may include an irreversible change in the physical properties such as the elasticity and viscosity of the mycelium mass. Texturization can lower protein solubility, increase digestibility, deactivate heat-sensitive growth inhibitors, and reduce the microbial load of the mycelium mass. Holding a shape of the mycelium mass in the container may lead to its texturization as the mycelium mass transforms from a low strength compacted structure to a continuously solid structure. The solid structure may be more elastic after the texturization which leads to a closer resemblance to an animal muscle product. The structure after texturization may also be harder, chewier, more tender, and juicier. The texturization process may also remove and/or reduce undesired flavors such as by destroying undesired enzymes, such as hydrolytic enzymes. The texturization process may also eliminate a rehydration step in typical processes for texturizing mycelium products which may result in case in manufacturing and scalability.

The container and/or mycelium mass may then be heated to a second temperature in a range from about 50° C. to about 100° C. (e.g., 50° C., 60° C., 70° C., 80° C., 90° C., or 100° C., inclusive). Heating the container to a temperature higher than 100° C. may result in the introduction and/or formation of undesired flavors. The container may be heated to the second temperature for a first period of time in a range from about 1 to about 120 minutes (e.g., 1 minute, 10 minutes, 20 minutes, 30 minutes, 40 minutes, 50 minutes, 60 minutes, 70 minutes, 80 minutes, 90 minutes, 100 minutes, 110 minutes, or 120 minutes, inclusive). In some embodiments, the second temperature is about 65° C., and the first period of time is about 30 minutes.

In some embodiments, a higher temperature may result in a shorter period of time in which the heat is applied. Heating the container may reduce the amount of time the vacuum is applied. In the texturization process without heat, the vacuum may be applied for a vacuum period of time in a range from about 18 to about 48 hours (e.g., 18 hours, 24 hours, 30 hours, 36 hours, 42 hours, or 48 hours, inclusive). The texturization process in which both the heat and the vacuum are applied may result in a shorter period of time in a range from about 1 to about 120 minutes (e.g., 1 minute, 10 minutes, 20 minutes, 30 minutes, 40 minutes, 50 minutes, 60 minutes, 70 minutes, 80 minutes, 90 minutes, 100 minutes, 110 minutes, or 120 minutes, inclusive).

In some embodiments, the container may be disposed in a water bath (e.g., a circulating water bath, a non-circulating water bath, a shaking water bath, or any other suitable water bath) heated to the second temperature. In such embodiments, the container may include plastic. In some embodiments, the container is a plastic bag, and the vacuum is applied to the plastic bag before disposing the container in the water bath.

In some embodiments, a wall of the container includes at least one resistive element configured to heat the container. A resistive element may heat the container by converting electrical energy from an electric current into heat. Resistive elements may be metal (e.g., Nichrome, Kanthal, cupronickel, etched foil, or any other suitable metal), ceramic or semiconductor (e.g., molybdenum disilicide, silicon carbide, silicon nitride, PTC ceramic elements, quartz halogen, or any other suitable ceramic or semiconductor), polymer (e.g., polymer PTC rubber materials or any other suitable polymer), or any other suitable material. In such embodiments, the container may be the mold. The container may be heated in any suitable process such as, but not limited to, a heat source of hot air, hot water, hot oil, electromagnetic waves, a dielectric oven, steam, convection, infrared radiation, any other suitable process, or combination thereof. A direct exposure to the source of heat may lead to a slow disintegration of the mycelium mass which was undesired. Therefore, heat may be applied through the container instead of directly.

In some embodiments, the mycelium mass may be mixed with food additives or colors, at 110. Adding at least one food additive or color to the mycelium mass 110 may be optional. Additives can include vegetable or animal proteins, fats, emulsifiers, thickeners, stabilizers, flavoring, fiber, gums, minerals, any other suitable additive, or combinations thereof. The colors can include any suitable color additive such as liquids, powders, gels, and pastes and may provide a similar color to meat products. The food additives or colors may be uniformly distributed in the mycelium mass or the texturized mycelium product. Adding at least one food additive or color to the mycelium mass may occur at any point in the method 100, including before and/or after applying the vacuum 106 and/or heating the container 108. In some embodiments, the food additives or colors may be added to mycelium mass disposed in the container before the vacuum is applied. In some embodiments, the food additives or colors may be added to mycelium mass disposed in the container before the vacuum and/or compression is applied or before a dewatering operation. In such embodiments, the texturization process may result in the uniform distribution of the food additives or colors throughout the texturized mycelium product. In some embodiments, the food additives or colors may be added to the texturized mycelium product after the vacuum and/or heat is applied.

A flavorant may be added to the resulting texturized mycelium product. The flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

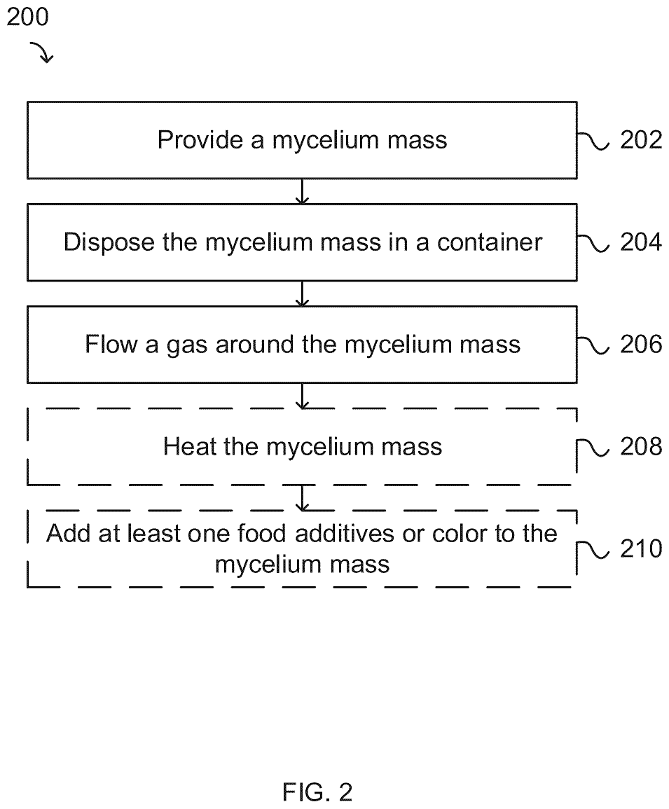

FIG. 2 illustrates a block diagram of an example method 200 for forming an edible meat substitute product, according to an embodiment. The method 200 may be substantially similar to the method 100, however may include flowing a gas around the mycelium mass in place of applying a vacuum to the mycelium mass. In a brief overview, the method 200 may include providing a mycelium mass, at 202. The method 200 may include disposing the mycelium mass in a container, at 204. The method 200 may include flowing a gas around the mycelium mass, at 206. The method 200 may include heating the mycelium mass, at 208. The method 200 may include adding at least one food additives or color to the mycelium mass, at 210.

The mycelium mass provided at 202 may be substantially similar to the mycelium mass provided at 102 in the method 100. Then, the mycelium mass at 202 may be disposed in a container, at 204, which may be substantially similar to the container discussed at 104 in the method 100.

The method 200 may then include flowing a gas around the mycelium mass, at 206. The gas may be at least one of air, carbon dioxide, or nitrogen. The gas may be from any suitable source, such as a commercially-available source, an atmospheric source, an industrial source, or a liquefied source. In some embodiments, the mycelium mass may be placed in an enclosed system in which the gas is flowed. In other embodiments, the container is enclosed, and the gas may enter the container and flowed around the mycelium mass. In such embodiments, the gas may enter the container through any suitable method, such as from a gas manifold, through a valve (e.g., an air actuated ball valve, a hand valve, a gate valve, a globe valve, a plug valve, a butterfly valve, a check valve, a diaphragm valve, a pinch valve, a pressure relief valve, a control valve, or any other suitable valve), or through a pump (e.g., a rotary vane pump, a diaphragm pump, a piston pump, a scroll pump, a screw pump, a Wankel pump, an external vane pump, any positive displacement pump, or any other suitable pump). The valve or the pump may be fluidly coupled with the container. The gas may be flowed to the container for a first gas time period in a range from about 1 to about 150 minutes (e.g., 1 minutes, 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, or 150 minutes, inclusive). The flowed gas may arrest the growth and/or metabolism of the mycelium mass disposed in the container, reducing the development of undesired flavors.

The method 200 may then including heating the mycelium mass to a first temperature and optionally, a second temperature, to form the texturized mycelium product, at 208. The operation 208 may be substantially similar to the operation 108 of method 100. The texturized mycelium product may have a chewiness in a range from about 500 to about 4500 g/cm² (e.g., 500 g/cm², 1000 g/cm², 1500 g/cm², 1600 g/cm², 1700 g/cm², 1800 g/cm², 1900 g/cm², 2000 g/cm², 2100 g/cm², 2200 g/cm², 2300 g/cm², 2400 g/cm², 2500 g/cm², 3000 g/cm², 3500 g/cm², 4000 g/cm², or 4500 g/cm², inclusive), such as from 1500 g/cm² to 2000 g/cm² or from 1800 g/cm² to 2000 g/cm². A mycelium mass that does not undergo texturization may have a chewiness in a range from about 1000 to about 1500 g/cm² (e.g., 1000 g/cm², 1100 g/cm², 1200 g/cm², 1300 g/cm², 1400 g/cm², or 1500 g/cm², inclusive) in comparison and/or may have a greater firmness and/or higher crumbliness compared to a mycelium mass that has undergone texturization. The texturized mycelium product may have a reduced firmness compared to the material before heat treatment. For example, in some embodiments, the firmness of the texturized mycelium product may be from about 40% to about 60% of the firmness of the mycelium mass before heat treatment. In some embodiments, an untreated mycelium mass may have a firmness of greater than or equal to 35 kg, and the texturized mycelium product may have a firmness that is in a range from about 15 kg to about 20 kg.

Flowing the gas to the mycelium mass and/or heating the container to the first temperature may form a texturized mycelium product and texturize the mycelium mass. Texturization may include an irreversible change in the physical properties such as the elasticity and viscosity of the mycelium mass. Texturization can lower protein solubility, increase digestibility, deactivate heat-sensitive growth inhibitors, and reduce the microbial load of the mycelium mass. Holding a shape of the mycelium mass in the container may lead to its texturization as the mycelium mass transforms from a low strength compacted structure to a continuously solid structure. The solid structure may be more elastic after the texturization which leads to a closer resemblance to an animal muscle product. The structure after texturization may also be harder, chewier, tenderer, and juicier. The texturization process may also remove undesired flavors such as destroying undesired enzymes. The texturization process may also eliminate a rehydration step in typical processes for texturizing mycelium products which may result in case in manufacturing and scalability. Then, the mycelium mass may be mixed with food additives or colors, at 210. The operation 210 may be substantially similar to the operation 110 of the method 100.

A flavorant may be added to the resulting texturized mycelium product. The flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

In some embodiments, a pasteurization process may be applied to the texturized mycelium product following 102, 104, and 106 of method 100 or 202, 204, and 206 of method 200. Pasteurization may be applied in order to partially sterilize the texturized mycelium product to make it safe for consumption and improve its keeping quality. In such embodiments, the container may be heated to the second temperature in a range from about 50° C. to about 100° C. (e.g., 50° C., 60° C., 70° C., 80° C., 90° C., or 100° C., inclusive) for a first period of time in a range from about 1 to about 120 minutes (e.g., 1 minute, 10 minutes, 20 minutes, 30 minutes, 40 minutes, 50 minutes, 60 minutes, 70 minutes, 80 minutes, 90 minutes, 100 minutes, 110 minutes, or 120 minutes, inclusive). In such embodiments, the texturized mycelium product can be considered pasteurized.

In such embodiments, the container may be heated to a third temperature. In some embodiments, the third temperature is the same as the second temperature. In some embodiments, the third temperature is greater than the second temperature, for example, in a range from about 60° C. to about 80° C. (e.g., 60° C., 65° C., 70° C., 75° C., or 80° C., inclusive). In such embodiments, the container may be heated for a second period of time in a range from about 30 to about 90 minutes (e.g., 30 minutes, 40 minutes, 50 minutes, 60 minutes, 70 minutes, 80 minutes, or 90 minutes, inclusive). Heating the container to the third temperature for the second period of time may result in pasteurization of the texturized mycelium product. In other words, the texturized mycelium product may be heated to the second temperature after the vacuum is applied, removed from the heat source and cooled, and subsequently heated to the third temperature, for example, to texturize the mycelium mass. In some embodiments, the container may be opened and at least one food additives and/or colors may be added to the texturized mycelium product after heating to the second temperature and before heating to the third temperature. In such embodiments, the texturized mycelium product can be considered pasteurized.

In some embodiments, the container may be disposed in the water bath heated to the second temperature and/or the third temperature. In such embodiments, the container may include plastic. In some embodiments, the container is the plastic bag, and the vacuum is applied to the plastic bag before disposing the container in the water bath.

In some embodiments, the wall of the container includes at least one resistive element configured to heat the container to the second temperature and/or the third temperature. In such embodiments, the container may be a mold. The container may be heated in any suitable process such as, but not limited to, hot air, hot water, hot oil, electromagnetic waves, steam, convection, infrared radiation, any other suitable process, or combination thereof. In some embodiments, the same process is used to heat the texturized mycelium product to the second temperature and the third temperature. In some embodiments, different processes are used to heat the texturized mycelium product to the second temperature and the third temperature. A direct exposure to the source of heat may lead to a slow disintegration of the texturized mycelium product which is undesired. Therefore, heat may be applied through the container instead of directly.

The pasteurization process may include 110 in which food additives or colors are added to the texturized mycelium product or the mycelium mass. The food additives or colors may be added at any point in the pasteurization process or the method 100. The food additives or colors may be uniformly distributed throughout the texturized mycelium product. In other embodiments, increasing the second temperature or lengthening the time that the container is heated at the second temperature without heating the texturized mycelium product to the third temperature results in the pasteurization of the texturized mycelium product. In such embodiments, the texturized mycelium product can be considered pasteurized.

A flavorant may be added to the resulting texturized mycelium product. The flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, barbeque powder, sour cream powder, lemon powder, lime powder, etc.).

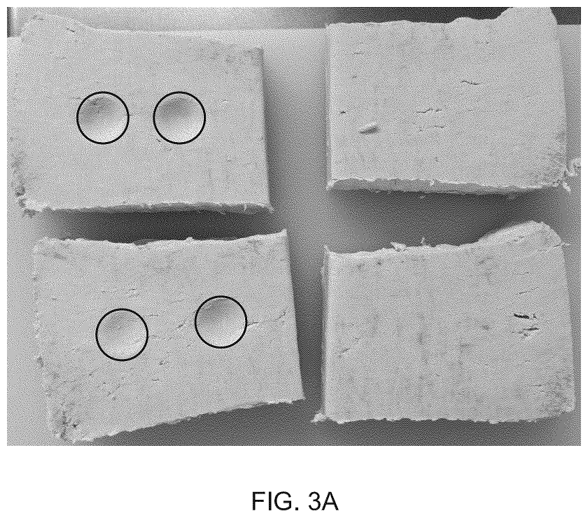

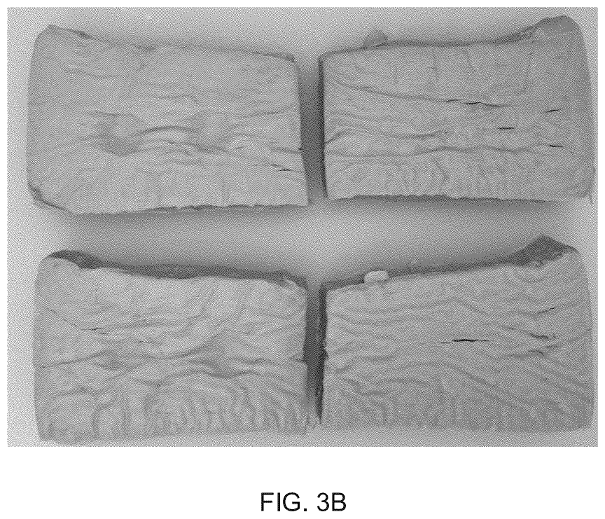

FIG. 3A is an image of a mycelium mass before texturization, in accordance with some embodiments. Indentations caused by texture profile analysis (TPA) probe tips are shown circled. The mycelium mass before texturization may be easier to deform upon compression than the resulting texturized mycelium.

FIG. 3B is an image of a texturized mycelium product formed from the mycelium mass after texturization, in accordance with some embodiments. The texturized mycelium product may be formed from the method 100 or the method 200. Texture profile analysis (TPA) probe tips may leave no indentations after the texturization process, demonstrating clastic behavior in the texturized mycelium product. Therefore, there may be an irreversible change in the physical properties as the mycelium mass transforms from viscous to elastic.

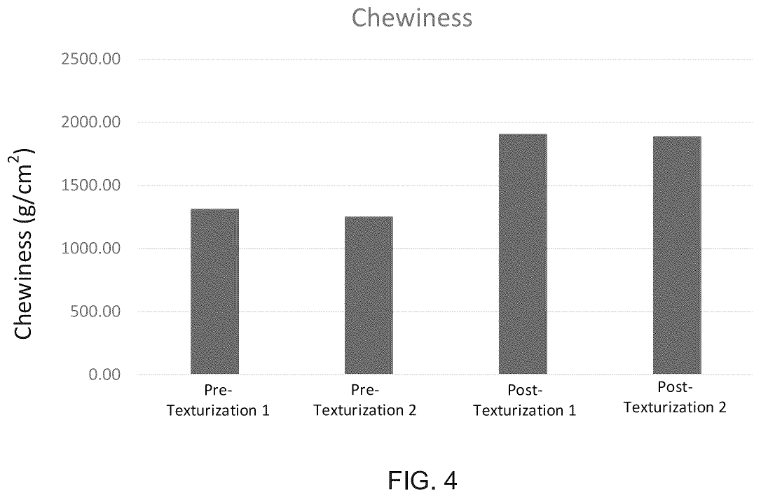

FIG. 4 is a plot of chewiness of the mycelium mass from FIGS. 3A-3B before and after texturization, in accordance with some embodiments. Chewiness is measured via instrumental analysis from a texture profile analysis (TPA) probe tip and using Texture Analyzer TA.XT. TPA is a measurement technique that is well known in the art and widely used for measuring various parameters of food products, including, but not limited to, hardness, cohesiveness, springiness, resilience, and chewiness. In a typical TPA analysis, a test sample of a food product is compressed using a texture analyzer such as a probe. One understanding of the chewiness parameter of a food product is the energy needed to chew a solid food until it is ready to be swallowed. Under that understanding, chewiness can be calculated as the product of the hardness, cohesiveness, and springiness parameters of a food product, where each of hardness, cohesiveness, and springiness is another parameter measurable using TPA. Before texturization, the mycelium mass is labeled “Pre-Texturization 1” and “Pre-Texturization 2.” After texturization, the texturized mycelium product is labeled “Post-Texturization 1” and “Post-Texturization 2.” As shown in FIG. 4, chewiness before texturization is in a range from about 1000 g/cm² and 1500 g/cm². After texturization, chewiness is in a range from about 1500 g/cm² and 2500 g/cm² demonstrating a substantial increase over the pre-texturization chewiness.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an”, and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

The disclosure is further illustrated by means of the following non-limiting example.

### Example 1: Firmness Measurement

Sample Preparation: Four samples of raw mycelium mass were prepared according to methods disclosed in U.S. Patent Application Publication No. 2019/0373935 A1, with the samples having a moisture content of 75%. Four samples of texturized mycelium product were prepared according to methods described herein.

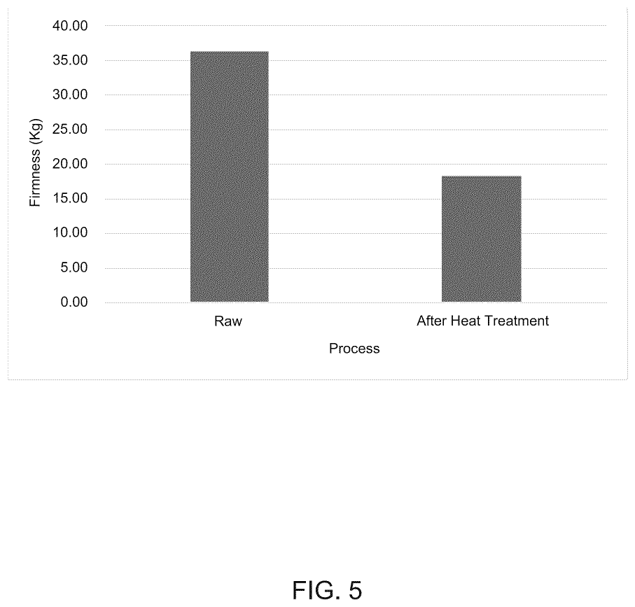

Testing: The firmness of the four samples of raw mycelium mass and the firmness of the four samples of texturized mycelium product were measured using standard approaches using a texture analyzer (TA.XTPlus, Texture Technologies, Hamilton, MA) equipped with a 4″ diameter, 10 mm tall, aluminum cylinder (TA-40). Each sample was analyzed at room temperature (55 to 77° F.). A 50 kg load cell at a test of speed of 1 mm/sec (pre-test), 1 mm/sec (test), 10 mm/sec (post-test) was used to reach a 40% compression with 50 g of trigger force. The results of the analysis are depicted in FIG. 5, which shows that the texturized mycelium product has a decreased firmness value compared to the raw mycelium mass.

What is claimed is:

1. A method of preparing an edible meat substitute product, comprising:
providing a mycelium mass;
disposing the mycelium mass in a container; applying a vacuum to the container; and
heating the mycelium mass to a temperature suitable to provide a texturized mycelium product.

2. The method of claim 1, further comprising:
after said heating, heating the mycelium mass to a second temperature in a range from 50° C. to 100° C.

3. The method of claim 2, wherein the mycelium mass is heated to the second temperature for a period of time in a range from about 1 minute to about 120 minutes.

4. The method of claim 2, wherein heating the mycelium mass to the second temperature comprises disposing the container in a liquid bath, a steam oven, a microwave oven, or an infrared oven that is heated to the second temperature.

5. The method of claim 1, wherein the container comprises plastic or metal.

6. The method of claim 1, wherein the container is enclosed.

7. The method of claim 1, further comprising:
adding at least one of food additives or colors to the mycelium mass.

8. The method of claim 7, wherein the at least one of food additives or colors is uniformly distributed in the texturized mycelium product.

9. The method of claim 1, wherein the product is pasteurized.

10. The method of claim 1, further comprising:
heating the mycelium mass to a second temperature in a range from 25° C. to 100° C. for a first period of time in a range from about 1 minute to about 120 minutes;
adding at least one of food additives or color to the texturized mycelium product; and
heating the mycelium mass to a third temperature in a range from 25° C. to 100° C., for a second period of time in a range from about 1 minute to about 120 minutes.

11. The method of claim 10, wherein the product is pasteurized.

12. The method of claim 1, wherein the mycelium mass is heated to a temperature in a range from about 25° C. to about 50° C.

13. The method of claim 1, wherein the texturized mycelium product has a chewiness in a range from about 1500 g/cm² to about 2000 g/cm² and/or a firmness that is in a range from about 15 kg to about 20 kg.

14. The method of claim 1, wherein the mycelium mass is a fungal mycelium comprising fungi from the genus Neurospora.

15. The method of claim 1, wherein the mycelium mass is a fungal mycelium comprising fungi from the species Neurospora crassa.

16. The method of claim 1, wherein the mycelium mass has a vitamin D content of from about 1 microgram per 100 grams to about 1,000 micrograms per 100 grams.

17. The method of claim 1, wherein the mycelium mass has an iron content of from about 5 mg of iron per 100 g of mycelium mass to about 15 mg of iron per 100 g of mycelium mass.

18. A method of preparing an edible meat substitute product, comprising:
providing a mycelium mass;
disposing the mycelium mass in a container;
flowing a gas around the mycelium mass; and
heating the mycelium mass to a temperature suitable to provide a texturized mycelium product.

19. The method of claim 18, further comprising:
heating the mycelium mass to a second temperature in a range of about 50° C. to about 100° C.

20. The method of claim 19, wherein the mycelium mass is heated to the second temperature for a period of time in a range from about 1 minute to about 120 minutes.

21. The method of claim 19, wherein heating the container to the second temperature comprises disposing the container in a liquid bath, a steam oven, a microwave oven, or an infrared oven that is heated to the second temperature.

22. The method of claim 18, wherein the container comprises plastic or metal.

23. The method of claim 18, wherein the container is enclosed.

24. The method of claim 18, further comprising:
adding at least one of food additives or colors to the mycelium mass.

25. The method of claim 24, wherein the at least one of food additives or colors is uniformly distributed in the texturized mycelium product.

26. The method of claim 18, wherein the product is pasteurized.

27. The method of claim 18, further comprising:
heating the mycelium mass to a second temperature in a range from about 25° C. to about 100° C. for a first period of time in a range from about 1 minute to about 120 minutes;
adding at least one of food additives or color to the texturized mycelium product; and
heating the mycelium mass to a third temperature in a range of about 25° C. to about 100° C., for a second period of time in a range from about 1 minutes to about 120 minutes.

28. The method of claim 27, wherein the product is pasteurized.

29. The method of claim 18, wherein the gas is at least one of air, carbon dioxide, or nitrogen.

30. The method of claim 18, wherein the mycelium mass is heated to a temperature in a range from about 25° C. to about 50° C.

31. The method of claim 18, wherein the texturized mycelium product has a chewiness in a range from about 1500 g/cm² to about 2000 g/cm² and/or a firmness that is in a range from about 15 kg to about 20 kg.

32. The method of claim 18, wherein the mycelium mass is a fungal mycelium comprising fungi from the genus Neurospora.

33. The method of claim 18, wherein the mycelium mass is a fungal mycelium comprising fungi from the species Neurospora crassa.

34. The method of claim 18, wherein the mycelium mass has a vitamin D content of from about 1 microgram per 100 grams about 1,000 micrograms per 100 grams.

35. The method of claim 18, wherein the mycelium mass has an iron content of from about 5 mg of iron per 100 g of mycelium mass to about 15 mg of iron per 100 g of mycelium mass.
